(19) United States
(12) Patent Application Publication
(10) Pub. No.: US 2012/0123421 A1
(43) Pub. Date: May 17, 2012

(54) CERAMIC CUTTIING TEMPLATE

(76) Inventors: Roman Preuss, Kirchheim unter Teck (DE); Heinrich Wecker, Eckental (DE); Matthias Eschle, Wannweil (DE)

(21) Appl. No.: 13/260,930

(22) PCT Filed: Apr. 1, 2010
(86) PCT No.: PCT/EP2010/054423

§ 371 Date: Sep. 29, 2011

(30) Foreign Application Priority Data
Apr. 1, 2009 (DE) 102009002085.3
Apr. 1, 2009 (DE) 102009002088.8

(51) Int. Cl. A61B 17/56; C04B 35/64; B28B 11/24; C04B 35/48

(52) U.S. Cl. 606/88; 501/134; 501/135; 501/87; 264/669; 264/604

(57) ABSTRACT

A cutting template or a cutting block, preferably to a cutting template or a cutting block for use in medical technology.

Subject matter of the present invention is a cutting template or a cutting block, preferably a cutting template or a cutting block for use in medical technology.

During each knee-TEP-implantation, a so-called cutting template or cutting block is fixed on the femur. With this cutting template, normally, three cuts are carried out for adapting the femur surface to the geometry of the femur component. For each cut, there is one guide in the cutting template (3 or 4 cutting guides in 1 template). In this guide, the cut is carried out with an oscillating saw blade. Today, saw blades and cutting templates are principally made of biocompatible metal alloys.

Depending on the manufacturer, the guide rails in the cutting block have a width of 1.2-1.5 mm. Due to the oscillation of the saw blade and the friction occurring between saw blade and guide rail, a significant metal abrasion on the guide rail occurs. This metal debris can not be removed intraoperatively or only insufficiently from the wound. Hence, this debris can become the cause of infections and, in particular, can result in allergic reactions in the patient. For this reason it is important to principally reduce said debris and in particular if an implant reaction by the use of a ceramic femur component in a potential allergy sufferer is to be avoided.

According to the current state of knowledge, the majority of the metal debris is generated through wear on the guide rails in the cutting template. After a cutting template has been used approximately 20-40 times during knee-TEP-implantations, the guide rails show guide gaps which are increased by approximately 0.5-1.5 mm. As a result, the guide accuracy of the cutting template decreases significantly. The consequences for the surgeon correspond; a precise cut of the saw blade is no longer possible, alignment and evenness of the cut surfaces of the femur deviate increasingly. This results in larger gaps between the cut surfaces and the femur component. Said gaps have to be filled intraoperatively by a volume of bone cement that is larger than the usual volume which can have a negative effect on the durability of the system.

The object underlying the present invention is to eliminate the disadvantages of the cutting templates/cutting blocks of the prior art and, in particular:
to reduce the metal debris, wherein a reduction the metal debris of up 90% with respect to previous metal solutions is be targeted;
to increase the service life of a cutting template and thus to save costs;
to reduce the risk of allergies and the risk of infections.

The object according to the invention was surprisingly achieved by a cutting template/a cutting block made of ceramics (hereinafter, the terms sinter-molded body or sintered body are also used for the cutting template according to the invention/cutting block according to the invention) with the features of the independent claims. Preferred configurations are to be found in the sub-claims. It was surprisingly found that the solution of the given object requires sinter-molded bodies with a very specific composition. Besides a transformation intensification achieved by embedding zirconium dioxide containing stabilizing oxides in a ceramic matrix, the invention provides as a matrix, according to a first embodiment, a mixed crystal from aluminum oxide/chromium oxide. The invention further provides that the zirconium dioxide embedded in the matrix and the chromium oxide, which, together with the aluminum oxide forms the mixed crystal, are in a defined molar relation to each other. This measure makes it possible that even in case of high zirconium dioxide proportions which can be required for maintaining particularly good fracture toughness, the required hardness values can be achieved. On the other hand, in case of low zirconium dioxide proportions, relatively low chromium oxide contents can be present, whereby an embrittlement of the material is counteracted.

The statement that the zirconium oxide containing the stabilizing oxides and the chromium oxide are to be present in a certain molar ratio results automatically in certain ratios for the other components because, e.g., with a decreasing proportion of zirconium oxide also the proportions of the stabilizing oxides, with respect to the sinter-molded body, decrease while, on the other hand, the proportion of the aluminum oxide increases. Based on the aluminum oxide of the sinter-molded body, the chromium oxide is present in a weight of 0.004 to 6.57 wt %, wherein, however, it should not be disregarded that the chromium oxide and the zirconium dioxide containing the stabilizing oxides are in the mentioned molar relation. Cerium oxide was found to be particularly advantageous as stabilizing oxide.

According to a further advantageous embodiment, the proportion of the matrix material in the sinter-molded body is at least 70 vol % and is formed from an aluminum oxide/chromium oxide mixed crystal with a chromium oxide proportion of 0.1 to 2.32 wt % based on aluminum oxide, wherein 2 to 30 vol % of zirconium oxide are embedded in the matrix, and the zirconium dioxide contains 0.27 to 2.85 mol % of yttrium oxide based on the mixture of zirconium oxide and yttrium oxide, and the zirconium oxide is present primarily in the tetragonal modification and has an average grain size not exceeding 2 μm. An amount of 0.27 to 2.85 mol % of yttrium oxide based on a mixture of zirconium dioxide and yttrium oxide corresponds to 0.5% to 5.4 wt % of yttrium oxide based on the zirconium dioxide. In case of such a sinter-molded body, the zirconium dioxide containing the yttrium oxide and the chromium oxide are present in a molar ratio of 370:1 to 34:1.

According to a further particularly preferred embodiment of the invention, the matrix material consists of an aluminum oxide/chromium oxide mixed crystal and a further mixed crystal with the formula SrAl₁₂₋ₓCrₓO₁₉, wherein x has a value of 0.0007 to 0.045. Also in this embodiment which, apart from that, corresponds to the first embodiment, the zirconium dioxide embedded in the mixed crystal matrix has a toughness-enhancing effect while the addition of chromium can counteract the decrease in hardness caused by the zirconium proportion. Surprisingly, it was found that in presence of strontium oxide, platelets are formed in the microstructure which platelets correspond to the formula SrAl₁₂₋ₓCrₓO₁₉. The mixed crystal with the formula SrAl₁₂₋ₓCrₓO₁₉ additionally formed by adding strontium oxide has the additional effect that it gives the sinter-molded body a further improved toughness even at higher temperatures. The wear resistance of these sinter-molded bodies under the influence of increased temperature is therefore also improved. In this embodiment too, the cerium oxide has proven to be particularly suitable. Platelets are formed even if the matrix contains no Cr₂O₃.

According to a further embodiment, the wear resistance of the sinter-molded bodies can be further improved by embedding therein 2 to 25 vol %—based on the matrix material—of one or a plurality of carbides, nitrides or carbonitrides of the metals of the 4^{th} and 5^{th} subgroup of the periodic table of elements. Preferably, the proportion of these hard materials is approximately 6 to 15 vol %. Particularly suitable are titanium nitride, titanium carbide and titanium carbonitride.

According to a particularly preferred further embodiment of the invention, the molar ratio of the zirconium dioxide containing the stabilizing oxides to chromium oxide is set depending on the zirconium dioxide present in the sinter-molded body according to the invention in such a manner that in case of low zirconium dioxide proportions, the chromium oxide quantities are low as well. It was found to be particularly suitable if the setting of the molar ratio of zirconium dioxide chromium oxide lies in the range
2-5 vol % of zirconium dioxide 1,000:1 to 100:1>5-15 vol % of zirconium dioxide 200:1 to 40:1>15-30 vol % of zirconium dioxide 100:1 to 20:1>30-40 vol % of zirconium dioxide 40:1 to 20:1.

In order to ensure that the zirconium dioxide is primarily present in the tetragonal modification it is required according to the invention to set a zirconium dioxide grain size not exceeding 2 μm. Besides the proportions of zirconium dioxide in cubic modification which are allowed up to an amount of 5 vol %, small amounts of the monoclinic modification are also allowed; however, they too are not to exceed an amount of max. 5 vol % and are preferably less than 2 vol %, particularly preferred even less than 1 vol % so that preferably more than 90 vol % are present in the tetragonal modification.

Since apart from the components stated in the patent claims, the sinter-molded body contains in addition only impurities introduced in an unavoidable manner which, according to another preferred embodiment, are not more than 0.5 vol %, the sinter-molded body consists only of the aluminum oxide-chromium oxide mixed crystal or, in presence of strontium oxide and chromium oxide, of this mixed crystal and the mixed crystal with the formula SrAl₁₂₋ₓCrₓO₁₉ and of the zirconium dioxide which contains the stabilizing oxides and is embedded in the matrix of the mentioned mixed crystals. Further phases such as, e.g., grain boundary phases which are formed when aluminum oxide and magnesium oxide are used together, or further crystalline phases which are generated by adding substances such as YNbO₄ or YTaO₄ which are known from the prior art and which have a softening point that is not high enough, are not present in the sinter-molded body according to the invention. Also, the oxides of Mn, Cu, and Fe which are known from the prior art and which also result in the formation of further phases cause a lowered softening point and lead to a low edge strength. The use of these materials is therefore excluded in the present invention.

Preferably, the zirconium dioxide is present in an amount of not more than 30 vol %. Preferably, the zirconium dioxide is also not present in an amount of less than 15 vol %. If between 15 and 30 vol % of zirconium oxide is present, the molar ratio between the zirconium dioxide containing the stabilizing oxides and the chromium oxide is particularly preferred between 40:1 and 25:1.

According to a further particularly preferred embodiment, the proportion of the zirconium dioxide present in tetragonal modification is more than 95 vol %, wherein only up to 5 vol % are present in total in the cubic and/or monoclinic modification. Particularly preferred is the compliance with a grain size of the embedded zirconium dioxide in the range of 0.2 to 1.5 μm. In contrast to that, an average grain size of the aluminum oxide/chromium oxide mixed crystal in the range of 0.8 to 1.5 μm was found to be particularly suitable. If in addition also carbides, nitrides and carbonitrides of the metals of the 4^{th} and 5^{th} subgroup of the periodic table of elements are used, they are used in a grain size of 0.8 to 3 μm. The grains of the mixed crystal with the formula SrAl₁₂₋ₓCrₓO₁₉ have a length/thickness ratio in the range of 5:1 to 15:1. Their maximum length is 12 μm and their maximum thickness is 1.5 μm.

It was surprisingly found that suitable platelets can be generated in the microstructure not only with strontium oxide but also with certain other oxides. A prerequisite for the platelet formation is the formation of a hexagonal crystal structure of the platelets to be formed. “in situ”. If the material system Al₂O₃—Cr₂O₃—ZrO₂—Y₂O₃ (CeO₂) is used as a matrix, the following platelets can be formed “in situ” with many different oxides. By adding alkali oxides, the corresponding alkali-Al₁₁₋ₓCrO₁₇ platelets are formed, by adding alkaline earth oxides, the corresponding alkaline earth-Al₁₂₋ₓCrₓO₁₉ platelets are formed, by adding CdO, PbO and HgO, the corresponding (Cd, Pb or HgAl₁₂₋ₓCrₓO₁₉) platelets are formed and by adding rare earth oxides, the corresponding rare earth-Al₁₁₋ₓCrₓO₁₈ platelets are formed. Moreover, La₂O₃ can form the compound La_{0.9}Al_{11.76-x}CrₓO₁₉. However, platelets are formed even if the matrix contains no Cr₂O₃. The platelets then forming without the presence of strontium oxide correspond to the general formulas: Alkali-Al₁₁O₁₇, alkaline earth-Al₁₂O₁₉, (Cd, Pb or HgAl₁₂O₁₉) or rare earth-Al₁₂O₁₈.

According to invention, the matrix material contains in a preferred configuration, an aluminum oxide/chromium oxide mixed crystal and a further mixed crystal according to one of the general formulas Me¹Al₁₁₋ₓCrₓO₁₇, Me²Al₁₂₋ₓCrₓO₁₉, Me^{2′}Al₁₂₋ₓCrₓO₁₉ or Me³Al₁₁₋ₓCrₓO₁₈, wherein. Me¹ represents an alkali metal, Me² represents an alkaline earth metal, Me^{2′} represents cadmium, lead or mercury and Me³ represents a rare earth metal, La_{0.9}Al_{11.76-x}CrₓO₁₉ can also be added as a mixed crystal to the matrix material. x can assume values ranging from 0.0007 to 0.045.

The “in situ” platelet reinforcement provided according to the invention occurs even if the matrix contains no Cr₂O₃. This is in particular provided according to the invention if a decrease of the hardness values is not disturbing. The platelets forming without Cr₂O₃ then correspond to the general formulas Me¹Al₁₁O₁₇, Me²Al₁₂O₁₉, Me^{2′}Al₁₂O₁₉ or Me³Al₁₂O₁₈. With these sinter-molded bodies too, the same preferred embodiments can be provided as with the sinter-molded bodies which contain Cr₂O₃ in the matrix material. In this respect, the explanations given above on the sinter-molded bodies containing Cr₂O₃ in the matrix material apply analogously to the sinter-molded bodies without Cr₂O₃ in the matrix material.

The Vickers hardness of the sinter-molded bodies according to the invention is greater than 1,750 [HV_{0.5}], but is preferably higher than 2,800 [HV_{0.5}].

The microstructure of the sinter-molded bodies according to the invention is free from micro-cracks and has a porosity degree of not more than 1%. The sinter-molded body can also contain whiskers, but not from silicon carbide.

The sinter-molded body preferably contains none of the substances often used as grain growth inhibitors such as, e.g., magnesium oxide.

The term “mixed crystal” used in the claims and the description is not to be understood in the meaning of single crystal; rather, a solid solution of chromium oxide in aluminum oxide or strontium aluminate is meant here. The sinter-molded body or the cutting template is polycrystalline.

During sintering, the stabilizer oxides in the ZrO₂ lattice disengage and stabilize the tetragonal modification of the latter. For producing the sinter-molded bodies and for achieving a microstructure free from further undesired phases, high-purity raw materials are used, i.e. aluminum oxide and zirconium oxide with a purity of greater than 99%. Preferably, the degree of impurities is significantly lower. In particular, SiO₂ proportions of greater than 0.5 vol % based on the finished sinter-molded body are undesirable. Excluded from this rule is the unavoidable presence of hafnium oxide in a small amount of up to 2 wt % within the zirconium dioxide.

Manufacturing the sinter-molded body is carried out by unpressurized sintering or hot pressing a mixture of aluminum oxide/zirconium dioxide/chromium oxide and stabilizing oxides, or a mixture of these components is used to which additionally also strontium oxide or instead of the strontium oxide, an alkali oxide, an alkaline earth oxide, CdO, PbO, HgO, a rare earth oxide or La₂O₃ and/or one or a plurality of nitrides, carbides and carbonitrides of the 4^{th} and 5^{th} subgroup of the periodic table of elements are added. Exemplary mixtures are specified in Table 1. The addition of yttrium oxide and chromium oxide can also take place in the form of yttrium chromium oxide (YCrO₃), whereas the addition of strontium oxide can preferably be carried out in the form of strontium salts, in particular in the form of strontium carbonate (SrCO₃). The alkali oxides, alkaline earth oxides, cadmium oxides, lead oxides, mercury oxides, rare earth oxides or the lanthanum oxide can preferably be added in the form of their salts, in particular in the form of carbonates. However, the addition of ternary compounds which disintegrate and reposition themselves during sintering is also possible. Different ceramic mixtures were produced by grinding. A temporary binder was added to the ground mixtures and subsequently, the mixtures were spray-dried. After this, the spray-dried mixtures were pressed into green bodies and sintered under standard conditions, for example sintered in an unpressurized manner or pre-sintered, and subjected to a gas pressure sintering process in an argon atmosphere.

The term unpressurized sintering comprises sintering under atmospheric conditions as well as under protective gas or in a vacuum. Preferably, the molded body is first pre-sintered without pressure to a theoretical density of 90 to 95% and subsequently re-densified by hot isostatic pressing or gas pressure sintering. The theoretical density can thereby be increased up to a value of more than 99.5%.

An alternative way of manufacturing the green body is achieved directly from the suspension. For this, a mixture with a solids content of more than 50 vol % is ground in an aqueous suspension. The pH value of the mixture is to be set to 4-4.5. After grinding, urea is added as well as an amount of the enzyme urease which is suited to degrade the urea before said suspension is poured into a mold. Due to the enzyme-catalyzed urea degradation, the pH value of the suspension shifts to 9, wherein the suspension coagulates. After demolding, the green body manufactured in this manner is dried and sintered. The sintering process can be carried out in an unpressurized manner, but pre-sintering followed by subsequent hot isostatic re-densification is also possible. Further details on this method (DCC method) are disclosed in WO 94/02429 and in WO 94/24064 to which express reference is made.

When manufacturing the ceramics on the basis of the mentioned multi-component systems, a number of factors can be of significant importance. In particular during the preparation of the powder mixture, dispersing and grinding can have a significant influence on the properties of the ceramics according to the invention. The grinding method and the grinding unit itself can have an impact on the result. Also, the solids content of the used grinding suspension can additionally contribute to the dispersion.

In the following examples, the influencing parameters and their effect on the mechanical properties are illustrated in more detail.

For the individual trials, the following combination of solids has been used

| | |
|---|---|
| Al₂O₃ | 73.11 wt % |
| ZrO₂ | 23.57 wt % |
| La₂O₃ | 2.48 wt % |
| YCrO₃ | 0.84 wt % |

For the trials V1-V2, a 60 wt % slurry has been used. In trial V5, the solids content was reduced to 55 wt %. For carrying out the trial V1, a vibrating tube mill was used. The trials V2 and V3 have been carried out using a laboratory attritor mill; the grinding time of V2 was 1 h, the grinding time of V3 was approximately 2 h. In trial. V4, a quantity of 30 kg has been processed in a continuous attritor mill. The trial V5 has been carried out in the laboratory attritor mill and a grinding duration of 2 h.

Below, the results from the strength tests for the individual trials are illustrated:

4-point bending strength

| | Average | | | Standard | |
|---|---|---|---|---|---|
| | [MPa] | min | max | deviation +/− | Weibullm |
| V1 | 692 | 480 | 835 | 105 | 7 |
| V2 | 789 | 297 | 942 | 162 | 4 |
| V3 | 1033 | 695 | 1243 | 113 | 10 |
| V4 | 1214 | 930 | 1373 | 93 | 15 |
| V5 | 997 | 781 | 1156 | 96 | 13 |

TABLE 1

| | Ex- | Ex- | Ex- | | | |
|---|---|---|---|---|---|---|
| | ample 1 | ample 2 | ample 3 | Example 4 | Example 5 | Example 6 |
| | [wt %] | [wt %] | [wt %] | [wt %] | [wt %] | [wt %] |
| Al₂O₃ | 73.30 | 58.62 | 73.60 | 84.16 | 66.95 | 63.53 |
| Cr₂O₃ | 0.86 | 1.20 | 0.40 | 0.10 | 0.86 | 0.78 |
| Oxide | 1.09* | 0.22** | 1.06* | 5.63*** | 0.95* | 1.06**** |
| ZrO₂ | 23.47 | 38.16 | 23.14 | 8.5 | 23.64 | 29.09 |
| Y₂O₃ | 1.28 | 1.80 | 0.13 | | 1.30 | |
| CeO₂ | | | 1.67 | 1.61 | | 5.54 |
| TiN | | | | | 6.3 | |

*La₂O₃;
**Er₂O₃;
***BaO;
****Dy₂O₃

With the teaching according to the invention, the metal debris is reduced by up to 90% compared to the previous cutting templates or cutting blocks made of metal. The service life of the cutting template or the cutting block according to the invention in use is considerably increased because only little wear on the cutting template occurs. This reduces the costs. Moreover, the allergy risk or the allergic reactions in patients and the risk of infections are reduced.

The cutting template is preferably used in the field of medical technology, in particular during surgeries for treating a bone, in a preferred manner during a knee-TEP-implantation.

The advantages of the ceramic cutting template or of the ceramics from which it is made are:
The cutting template shows extremely low abrasive wear.
The material is biocompatible.
If the cutting template is labeled by a laser, the template is clearly visible and readable and therefore can reduce wrong handling during the use of the cutting template.
The cutting template has very good tribological properties.

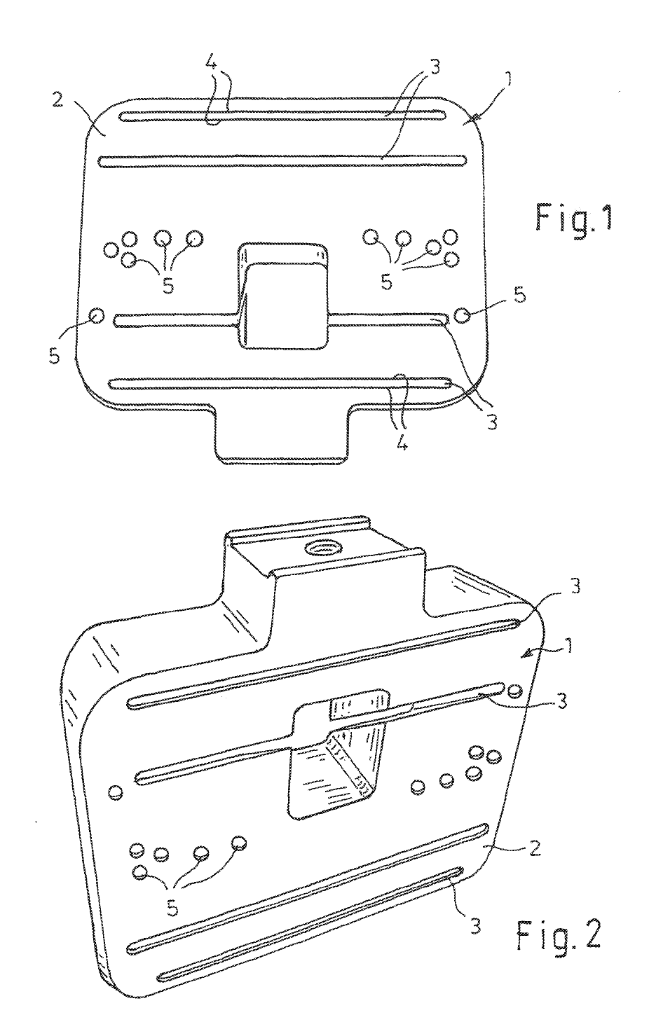

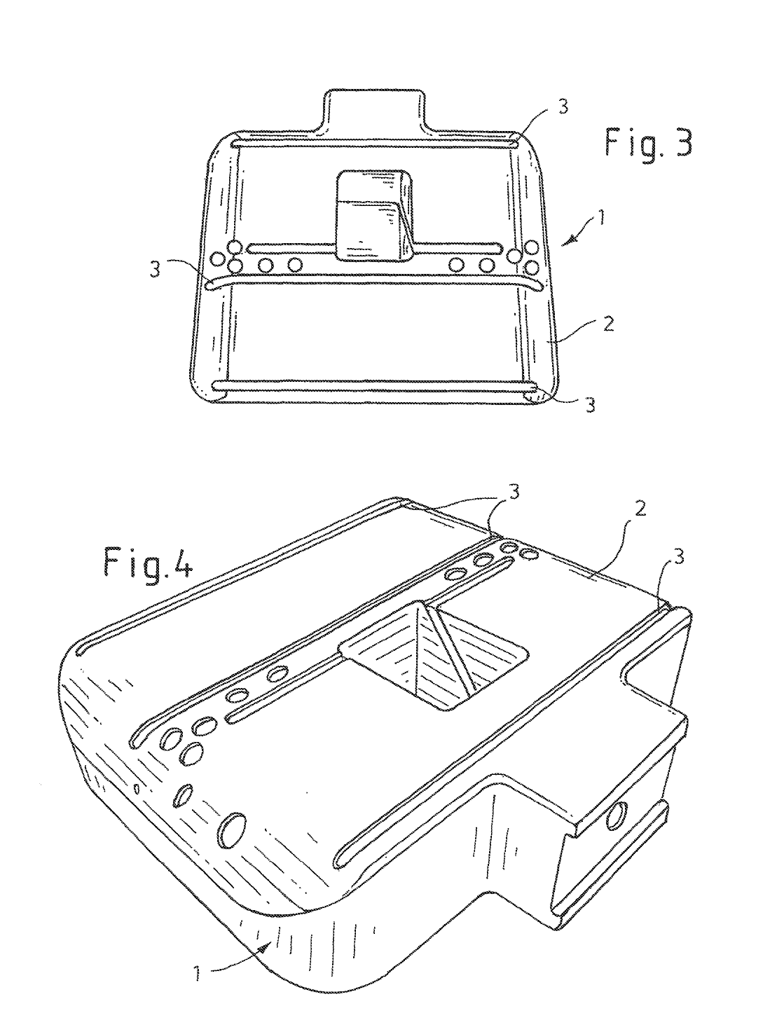

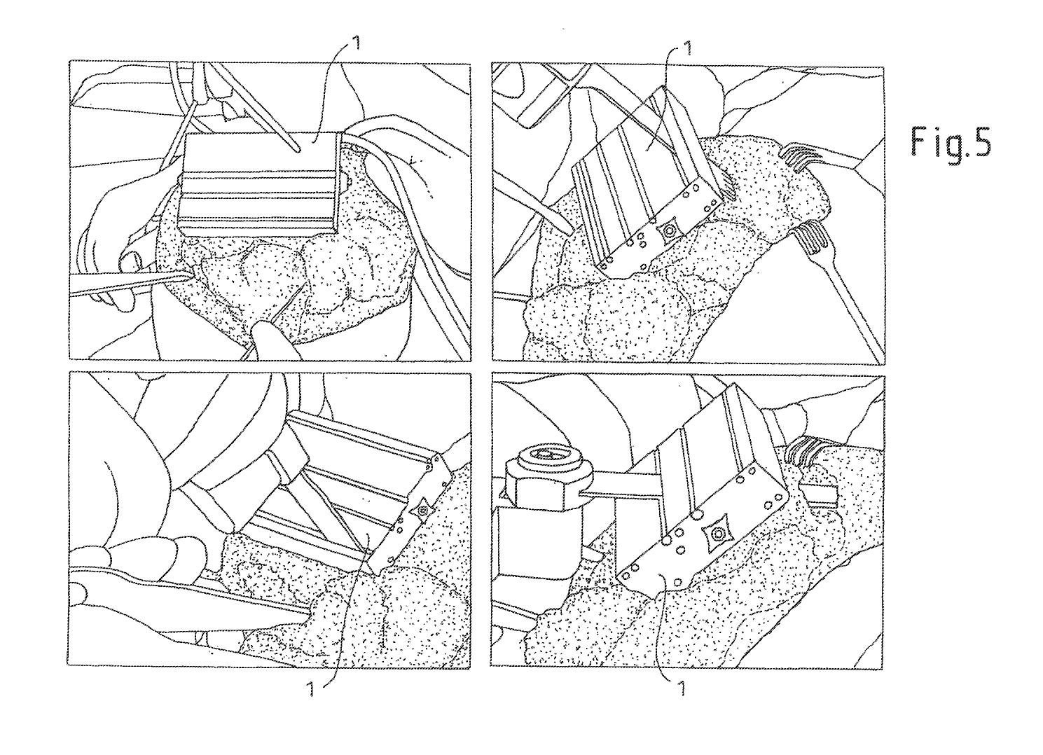

FIGS. 1 to 4 show a cutting template 1 according to the invention made of ceramics in different views. FIG. 5 shows images with respect to the shape and the intraoperative use of a conventional cutting template made of metal.

FIGS. 1 to 4 show a cutting template 1 according to the invention which is also designated as cutting block. Such a cutting template 1 serves for guiding a surgical saw blade during an implantation of an artificial knee joint.

The cutting template consists of a base body 2 which is provided with slot-like recesses 3 for inserting and precisely guiding a plate-shaped saw blade, wherein the slot-like recesses 3 have guide surfaces 4 which oppose each other. During the sawing process, the saw blade (see FIG. 5) rests against these guide surfaces 4. Through-holes 5 are drilled into the base body 2 which holes serve for screwing the cutting template 1 onto the femur.

Within the context of the present invention, the terms sinter-molded body/sintered body designate a ceramics in the form of a cutting template or cutting block or, respectively, a ceramics for the use as a cutting template or cutting block.

1. A cutting template, made from:
a) 60 to 98 wt % of a matrix material, formed from an aluminum oxide/chromium oxide mixed crystal,
b) 2 to 40 vol % of zirconium dioxide which is embedded in said matrix material and which
c) as stabilizing oxides, contains more than 10 to 15 mol % of one or a plurality of the oxides of cerium, praseodymium and terbium, based on the mixture of zirconium dioxide and stabilizing oxides, wherein
d) the added quantity of stabilizing oxides is selected such that the zirconium dioxide is present primarily in the tetragonal modification and
e) the molar ratio between the zirconium dioxide containing the stabilizing oxides and the chromium oxide is 1,000:1 to 20:1,
f) and the proportions of all components add up to 100 vol % of the sinter-molded body.

2. The cutting template, made from;
a) at least 70 vol % of a matrix material, formed from an aluminum oxide/chromium oxide mixed crystal with a chromium oxide proportion of 0.01 to 2.32 wt % based on aluminum oxide,
b) 2 to 30 vol % of zirconium dioxide which is embedded in the matrix material and which
c) contains 0.27% to 2.85 mol % of yttrium oxide based on the mixture of zirconium dioxide and yttrium oxide, wherein the added quantity of the yttrium oxide is selected such that the zirconium dioxide is present primarily in the tetragonal modification and
d) the molar ratio between the zirconium dioxide containing the stabilizing oxides and the chromium oxide is 1,000:1 to 20:1 and
e) the proportions of all components add up to 100 vol % of the cutting template.

3. The cutting template according to claim 2, wherein the molar ratio between the zirconium oxide containing the stabilizing oxides and the chromium oxide is 370:1 to 34:1.

4. The cutting template, made from
a1) 60 to 98 vol % of a matrix material, wherein the latter contains up to
a2) 67.1 to 99.2 vol % of an aluminum oxide/chromium oxide mixed crystal
a3) up to 0.8 to 32.9 vol % of a further mixed crystal which is selected from at least one mixed crystal according to one of the general formulas SrAl₁₂₋ₓCrₓO₁₉, La_{0.9}Al_{11.76-x}CrₓO₁₉, Me¹Al₁₁₋ₓCrₓO₁₇, Me²Al₁₂₋ₓCrₓO₁₉, Me^{2′}Al₁₂₋ₓCrₓO₁₉ and/or Me³Al₁₁₋ₓCrO₁₈, wherein Me¹ represents an alkali metal, Me² represents an alkaline earth metal, Me^{2′} represents cadmium, lead or mercury and Me³ represents a rare earth metal and x corresponds to a value of 0.0007 to 0.045 and
b) the matrix material contains 2 to 40 vol % of tetragonally stabilized zirconium dioxide which is embedded in the matrix material and
c) the proportions of the components add up to 100 vol % of the cutting template.

5. The cutting template according to claim 4, characterized in that as a stabilizing agent for the zirconium oxide, 2 to 15 mol % of one or a plurality of the oxides of cerium, praseodymium and terbium and/or 0.2 to 3.5 mol of yttrium oxide based on the mixture of zirconium dioxide and stabilizing oxides is used wherein the added quantity of stabilizing oxides is selected such that the zirconium oxide is present primarily in the tetragonal modification and the proportion of cubic modification is approximately 0 to 5 vol % based on the zirconium oxide.

6. The cutting template according to claim 4 or claim 5, characterized in that the molar ratio between the zirconium oxide containing the stabilizing oxides and the chromium oxide is 1,0001 to 20:1.

7. The cutting template according to one or more of the claims 1 to 6, characterized in that the zirconium dioxide has a grain size not exceeding 2 μm.

8. The cutting template according to one or more of the claims 1 to 7, characterized in that the matrix material contains in addition also 2 to 25 vol % of one or a plurality of the carbides, nitrides and carbonitrides of the metals of the fourth and fifth subgroup of the periodic table of elements based on the matrix material.

9. The cutting template, made from
a) 60 to 85 vol % of a matrix material, formed from an aluminum oxide/chromium oxide mixed crystal and from 2 to 25 vol % of one or a plurality of the carbides, nitrides and carbonitrides of the metals of the fourth and fifth subgroup of the periodic table of elements—based on the matrix material,
b) more than 15 to 40 vol % of zirconium dioxide which is embedded in the matrix material and which
c) as stabilizing oxides contains more than 10 to 15 mol % of one or a plurality of the oxides of cerium, praseodymium and terbium and/or 0.2 to 3.5 mol % of yttrium oxide based on the mixture of zirconium dioxide and stabilizing oxides, wherein
d) the added quantity of the stabilizing oxides is selected such that the zirconium dioxide is present primarily in the tetragonal modification and
e) the molar ratio between the zirconium dioxide containing the stabilizing oxides and the chromium oxide is 100:1 to 20:1,
f) the proportions of all components add up to 100 vol % of the sinter-molded body,
g) the zirconium dioxide has a grain size not exceeding 2 μm.

10. The cutting template according to any one of the claims 1 to 9, characterized in that the molar ratio of the zirconium dioxide containing the stabilizing oxides to the chromium oxide is in the range of
2-5 vol % of zirconium dioxide 1,000:1 to 100:1>5-15 vol % of zirconium dioxide 200:1 to 40:1>15-30 vol % of zirconium dioxide 100:1 to 20:1>30-40 vol % of zirconium dioxide 40:1 to 20:1.

11. The cutting template according to one or more of the claims 1 to 10, characterized in that not more than 30 vol % of zirconium dioxide are contained.

12. The cutting template according to one or more of the claims 1 to 11, characterized in that the zirconium dioxide has the tetragonal modification to at least 95 vol %.

13. The cutting template according to one or more of the claims 1 to 12, characterized in that the total zirconium dioxide content is present in the cubic and/or monoclinic modification in a proportion of 0 to 5 vol %.

14. The cutting template according to one or more of the claims 1 to 13, characterized in that the average grain size of the aluminum oxide/chromium oxide mixed crystal ranges from 0.6 to 1.5 μm.

15. The cutting template according to one or more of the claims 1 to 14, characterized in that the grain size of the zirconium dioxide ranges between 0.2 and 1.5 μm.

16. The cutting template according to one or more of the claims 1 to 14, characterized in that not more than 0.5 vol % of unavoidable impurities, based on the sinter-molded body, are contained.

17. The cutting template according to one or more of the claims 1 to 14, characterized in that the Vickers hardness [Hv 0.5]>1,800.

18. A cutting template comprising a matrix material, characterized in that the matrix material contains at least one of the platelets according to one of the general formulas SrAl₁₂₋ₓCrₓO₁₉, La_{0.9}Al_{11.76-x}CrₓO₁₉, Me¹Al₁₁O₁₇, Me²Al₁₂O₁₉, Me^{2′}Al₁₂O₁₉ and/or Me³Al₁₂O₁₈, wherein Me¹ represents an alkali metal, Me² represents an alkaline earth metal, Me^{2′} represents cadmium, lead or mercury and Me³ represents a rare earth metal and the matrix material contains tetragonally stabilized zirconium oxide.

19. A method for manufacturing a cutting template according to one or more of the claims 1 to 18, characterized in that a mixture containing aluminum oxide, zirconium oxide, chromium oxide, oxides stabilizing tetragonal zirconium oxide and at least one oxide selected from strontium oxide, alkali oxides, alkaline earth oxides, CdO, PbO, HgO, rare earth oxides and/or La₂O₃ is ground, a temporary binder is added to the mixture, this mixture is spray-dried, this mixture is pressed into green bodies and the latter are sintered under standard conditions.

20. The method according to claim 19, characterized in that the green body is pre-sintered in an unpressurized manner to a density of 90-95% and is subsequently subjected to a hot isostatic re-densification.

20. The method for manufacturing a cutting template according to one or more of the claims 1 to 18, characterized in that a mixture containing aluminum oxide, chromium oxide, tetragonal zirconium oxide, optionally stabilizing oxides and at least one oxide selected from strontium oxide, alkali oxides, alkaline earth oxides, CdO, PbO, HgO, rare earth oxides and/or La₂O₃ is ground in an aqueous suspension having a solids content of more than 50 vol % while maintaining a pH value of 4 to 4.5, subsequently, urea and urease are added thereto, is poured into a mold and is demolded after a subsequent coagulation and sintered or pre-sintered and hot-isostatically re-densified.

21. A use of the cutting template according to any one of the claims 1 to 18 in the field of medical technology, in particular during surgeries for treating a bone.

22. The use of the cutting template according to any one of the claims 1 to 18 during a knee-TEP-implantation.
